(19) United States
(12) Patent Application Publication
(10) Pub. No.: US 2009/0112205 A1
(43) Pub. Date: Apr. 30, 2009

(54) CARTRIDGE ELECTRODE DEVICE

(75) Inventors: Scott A. McGill, San Ramon, CA (US); Peter G. Knopp, Pleasanton, CA (US); Bankim H. Mehta, San Ramon, CA (US)

Correspondence Address: LEVINE BAGADE HAN LLP, 2483 EAST BAYSHORE ROAD, SUITE 100, PALO ALTO, CA, 94303, US

(73) Assignee: PRIMAEVA MEDICAL, INC., Fremont, CA (US)

(21) Appl. No.: 12/024,925

(22) Filed: Feb. 1, 2008

Related U.S. Application Data
(60) Provisional application No. 60/984,303, filed on Oct. 31, 2007.

(51) Int. Cl. A61B 18/14

(52) U.S. Cl. 606/41

(57) ABSTRACT

The invention provides a system and method for achieving the cosmetically beneficial effects of shrinking collagen tissue in the dermis or other areas of tissue in an effective, non-invasive manner using an array of electrodes. Systems described herein allow for improved treatment of tissue. Additional variations of the system include array of electrodes configured to minimize the energy required to produce the desired effect.

### CROSS-REFERENCE

This application is a non-provisional of U.S. Provisional Application No. 60/984,303 filed on Oct. 31, 2007 entitled “CARTRIDGE ELECTRODE DEVICE” the entirety of which is incorporated reference.

### BACKGROUND OF THE INVENTION

The systems and method discussed herein treat tissue in the human body. In a particular variation, systems and methods described below treat cosmetic conditions affecting the skin of various body parts, including face, neck, and other areas traditionally prone to wrinkling, lines, sagging and other distortions of the skin.

Exposure of the skin to environmental forces Can, over time, cause the skin to sag, wrinkle, form lines, or develop other undesirable distortions. Even normal contraction of facial and neck muscles, e.g. by frowning or squinting, can also over time form furrows or bands in the face and neck region. These and other effects of the normal aging process can present an aesthetically unpleasing cosmetic appearance.

Accordingly, there is well known demand for cosmetic procedures to reduce the visible effects of such skin distortions. There remains a large demand for “tightening” skin to remove sags and wrinkles especially in the regions of the face and neck.

One method surgically resurfaces facial skin by ablating the outer-layer of the skin (from 200 μm to 600 μm), using laser or chemicals. In time, a new skin surface develops. The laser and chemicals used to resurface the skin also irritate or heat the collagen tissue present in the dermis. When irritated or heated in prescribed ways, the collagen tissue partially dissociates and, in doing so, shrinks. The shrinkage of collagen also leads to a desirable “tightened” look. Still, laser or chemical resurfacing leads to prolonged redness of the skin, infection risk, increased or decreased pigmentation, and scarring.

Lax et al. U.S. Pat. No. 5,458,596 describes the use of radio frequency energy to shrink collagen tissue. This cosmetically beneficial effect can be achieved in facial and neck areas of the body in a minimally intrusive manner, without requiring the surgical removal of the outer layers of skin and the attendant problems just listed.

Utely et al. U.S. Pat. No. 6,277,116 also teaches a system for shrinking collagen for cosmetically beneficial purposes by using an electrode array configuration.

However, areas of improvement remain with the previously known systems. In one example, fabrication of an electrode array may cause undesired cross-current paths forming between adjacent electrodes resulting in an increase in the amount of energy applied to tissue.

In another example, when applying the array to tissue, the medical practitioner experiences a “bed-of-nails”. In other words, the number of electrodes and their configuration in the array effectively increases the total surface area of the electrode array. The increase in effective surface area then requires, the medical practitioner to apply a greater force to the electrode array in order to penetrate, tissue. Such a drawback may create collateral damage as one or more electrode may be placed too far within the skin. Additionally, the patient may experience the excessive force as the medical practitioner increases the applied force to insert the array within tissue.

Thermage, Inc. of Hayward Calif. also holds patents and sells devices for systems for capacitive coupling of electrodes to deliver a controlled amount of radiofrequency energy. This controlled delivery of RF energy creates an electric field through the epidermis that generates “resistive heating” in the skin to produce cosmetic effects while simultaneously attempting to cool the epidermis with a second energy source to prevent external burning of the epidermis.

In such systems that treat in a non-invasive manner, generation of energy to produce a result at the dermis results in unwanted energy passing to the epidermis. Accordingly, excessive energy production creates the risk of unwanted collateral damage to the skin.

In view of the above, there remains a need for an improved energy delivery system. Such systems may be applied to create improved electrode array delivery system for cosmetic treatment of tissue. In particular, such an electrode array may provide deep uniform heating by applying energy to tissue below the epidermis to cause deep structures in the skin to immediately tighten. Over time, new and remodeled collagen may further produce a tightening of the skin, resulting in a desirable visual appearance at the skin's surface. Such systems can also provide features that increase the likelihood that the energy treatment will be applied to the desired target region. Moreover, devices and systems having disposable or replaceable energy transfer elements provide systems that offer flexibility in delivering customized treatment based on the intended target tissue.

Moreover, the features and principles used to improve these energy delivery systems can be applied to other areas, whether cosmetic applications outside of reduction of skin distortions or other medical applications.

### SUMMARY OF THE INVENTION

The invention provides improved systems and methods of delivering energy to tissue. In one aspect of the invention, the methods and systems produce cosmetically beneficial effects of using energy to shrink collagen tissue in the dermis in an effective manner that prevents the energy from affecting the outer layer of skin.

In one variation, the systems and methods include a carrier and an array of electrodes on the carrier or handle, which are connectable to a source of electromagnetic energy to apply the electromagnetic energy. The devices and methods described herein can also be used to treat tissue masses such as tumors, varicose veins, or other tissue adjacent to the surface of tissue.

The devices and methods described herein may provide electrode arrays provided in a cartridge body that is removably coupled to a treatment device, where an electrode array of the cartridge device can penetrate tissue at an oblique angle or at a normal angle as discussed below. In addition, in those variations where the electrode array enters at an oblique angle, the device may include a cooling surface that directly cools the surface area of tissue adjacent to the treated region of tissue. The cooling methods and apparatus described herein may be implemented regardless of whether the electrodes penetrate at an oblique angle or not.

In one variation of the device, the device comprises: a device body having a handle portion, a cartridge receiving surface, an actuator adjacent thereto, and a plurality of electrically conductive leads on at least a portion of the cartridge receiving surface and being electrically coupleable to the energy source, where the actuator is moveable relative to the device body; a cartridge body removably coupled to the device body on the cart ridge receiving surface, the cartridge body comprising an electrode assembly in engagement with the actuator, the electrode assembly having a plurality of electrodes arranged in an array and at least one of the electrodes having a connection portion, the electrode assembly being moveable between a treatment position and a retracted position upon movement of the actuator, such that in the treatment position one or more electrodes can extend from the cartridge body and the respective connection portion engages one electrically conductive lead, and in the retracted position, one or more electrode retracts into the cartridge and the respective connection portion moves out of engagement with the electrically conductive lead preventing delivery of energy.

In additional variations, the cooling surface pre-cools the skin and underlying epidermis prior to delivering the therapeutic treatment. Additional variations include application of cooling during and/or subsequent to the energy delivery where such cooling is intended to minimize undesired damage to the epidermis, to maintain the epidermis temperature, and/or to retain the epidermis in a normal condition.

Variations of the invention include movement of the electrodes by use of a spring or other means to provide an impact force to the electrodes. The spring provides a spring force to move the electrodes at a velocity that allows, for easier insertion of the electrode array into tissue.

Alternatively, or in combination, the electrodes may be coupled to an additional source of energy that imparts vibration in the electrodes (e.g., an ultrasound energy generator). The same energy source may be used to generate the thermal effect in the dermis.

The methods and devices described herein may also use features to facilitate entry of the electrodes into tissue. For example, the surface tissue may be placed in traction prior to advancing electrodes through the surface tissue. The electrodes can comprise a curved shape. Where advancing the curved electrodes through tissue comprises rotating the electrodes into tissue.

The power supply for use with the systems and methods described herein may comprise a plurality of electrode pairs, each electrode pair comprising a mono-polar or bi-polar configuration. Each electrode pair of the system may be coupled to an independent channel of a power supply or independent power supplies. Such configurations permit improved controlled delivery of energy to the treatment site.

Another variation of the invention includes a cartridge and/or hand unit having any number of electronic storage units or memory (e.g., SRAM, DRAM, Masked ROM, PROM, EPROM, EEPROM, Flash memory, NVRAM, etc. or any combination thereof). Such memory capabilities can contain instructions or record communication between the cartridge and hand unit and/or controller to adjust treatment parameters, monitor usage, monitor sterility, or to record and convey other system or patient characteristics. In yet another variation, the cartridge and/or hand unit can include an RFID antenna/receiver configuration for preventing or permitting treatment given that the hand unit/controller recognizes a code embedded with the RFID antenna.

Another variation that controls delivery of energy may include spacing where each electrode pair is at a sufficient distance from an adjacent electrode pair to minimize formation of a cross-current path between adjacent electrode pairs. Moreover, the independent power supply can be configured to energize adjacent electrode pairs at different times.

It is expressly intended that, wherever possible, the invention includes combinations of aspects of the various embodiments described herein or even combinations of the embodiments themselves.

In addition, the concepts disclosed herein can be combined with the following commonly assigned applications where such combinations are possible: U.S. patent application Ser. No. 11/676,230 entitled “METHODS AND DEVICES FOR TREATING TISSUE filed on Feb. 16, 2007; PCT application No.: PCT/US2007/081556 entitled “METHODS AND DEVICES FOR TREATING TISSUE filed on Oct. 16, 2007;

U.S. patent application Ser. No. 11/764,032 entitled “METHODS AND DEVICES FOR TREATING TISSUE filed on Jun. 15, 2007; and U.S. patent application Ser. No. 11/832,544 entitled “METHODS AND DEVICES FOR TREATING TISSUE filed on Aug. 1, 2007. Each of which is incorporated by reference herein.

### BRIEF DESCRIPTION OF THE DRAWINGS

FIG. 1 shows a representative sectional view of skin and underlying subcutaneous tissue;

FIG. 2A shows a sample variation of a system according to the principles of the invention;

FIG. 2B illustrates a partial view of a working end of a treatment unit engaging tissue such that the array enters the tissue;

FIG. 3A illustrates a perspective view of a variation of a cartridge body for use with the present system;

FIG. 3B shows a cross sectional view of the cartridge body of FIG. 3A;

FIGS. 3C to 3E show a cross sectional view of a cartridges body here the electrodes are offset to prevent the formation of a continuous line of insertion marks in the tissue;

FIG. 4A illustrates a cross sectional view of a variation of a treatment unit.

FIG. 4B shows a partial perspective cross sectional view of the treatment unit of FIG. 4A;

FIG. 4C shows a side cross sectional view of a working end of a treatment unit and cartridge coupled together:

FIGS. 4D and 4E illustrates a variation of multiple conductive leads on a connection board for selective coupling to multiple conductive leads on an electrode;

FIG. 5 shows a graph representing pulsed energy delivery and temperature measurements between pulses of energy;

FIGS. 6A to 6B show variations of introducer members that assist in placing electrodes within tissue;

FIG. 7A shows an additional variation of a device having an array of electrodes in a removable cartridge adjacent to a tissue engaging surface;

FIG. 7B shows a magnified View of the electrodes and tissue engaging surface of the device of FIG. 7A;

FIG. 7C shows an example of an electrode entering tissue at an oblique angle adjacent to a tissue engaging surface;

FIG. 8 shows another example of an electrode entering tissue at an oblique angle underneath a skin anomaly:

FIG. 9A to 9C show cooling surfaces adjacent to the electrodes;

FIGS. 10A-10D illustrate variations of electrodes having varying resistance or impedance along the length of the electrode; and

FIGS. 11A to 11B show an example of an array of electrodes where any number of pairs of electrodes can be triggered to apply therapeutic energy to tissue.

### DESCRIPTION OF THE PREFERRED EMBODIMENT

The systems and method discussed herein treat tissue in the human body. In one variation, the systems and methods treat cosmetic conditions affecting the skin of various body parts, including face, neck, and other areas traditionally prone to wrinkling lines, sagging and other distortions of the skin. The methods and systems described herein may also have application in other surgical fields apart from cosmetic applications.

The inventive device and methods also include treatment of skin anomalies, such as, warts (Verruca plana, Verruca vulgaris), sebaceous hyperplasia or acne (Acne vulgaris). Treatment of acne can be accomplished by the direct ablation of sebaceous glands or it can be accomplished by the delivery of thermal energy which will stimulate the body's immune system to eliminate the bacteria, Propionibacterium acnes, which is one of the causes of acne. The methods and devices can be used for the removal of unwanted hair (i.e., epilation) by applying energy or heat to permanently damage hair follicles thereby removing the skins ability to grow hair. Such treatment may be applied on areas of facial skin as well as other areas of the body.

Other possible uses include pain management (both in the use of heat to reduce pain in muscle tissue and by directly ablating nociceptive pain fibers), stimulation of cellular healing cascade via heat, treatment of the superficial muscular aponeurotic system (SMAS), reproductive control by elevated heating of the testicles, and body modification such as piercing, scarification or tattoo removal

In addition to therapeutic surface treatments of the skin, the current invention can be targeted to the underlying layer of adipose tissue or fat for lipolysis or the breakdown of fat cells. Selecting electrodes having sufficient length to reach the subcutaneous fat layer allows for such electrodes to apply energy in the subcutaneous fat layer. Application of the energy can break down the fat cells in that layer allowing the body to absorb the resulting free fatty acids into the blood stream. Such a process can allow for contouring of the body surface for improved appearance. Naturally, such an approach can be used in the reduction of cellulite.

Other possible uses include pain management (both in the use of heat to reduce pain in muscle tissue and by directly ablating nociceptive pain fibers), stimulation of cellular healing cascade via heat, reproductive control by elevated heating of the testicles, and body modification such as scarification.

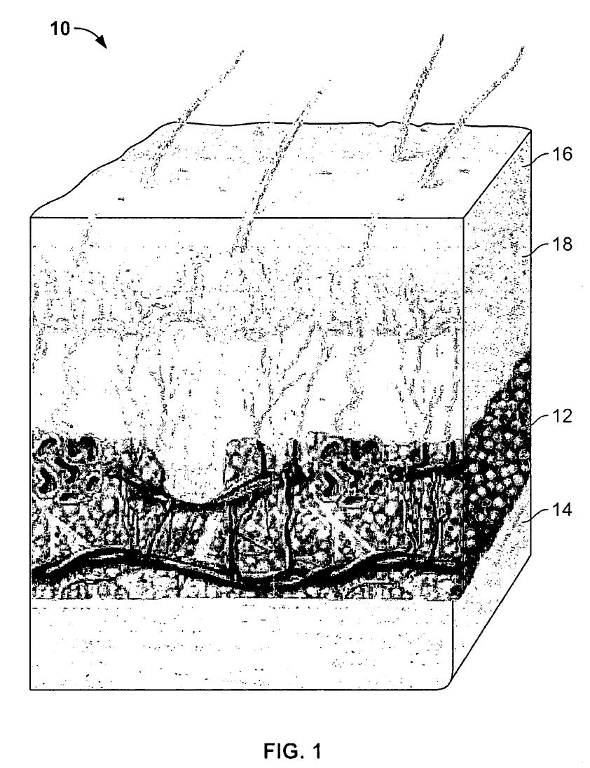

As FIG. 1 shows, the skin 10 covers-subcutaneous tissue 12 and muscle tissue 14 of within the body. In the face and neck areas, the skin 10 measures about 2 min in cross sectional depth.

The skin 10 includes an external, non-vascular covering called the epidermis 16. In the face and neck regions, the epidermis measures about 100 μm in cross sectional depth. The skin 10 also includes a dermis 18 layer that contains a layer of vascular tissue. In the face and neck regions, the dermis 18 measures about 1900 μm in cross sectional depth.

The dermis 18 includes a papillary (upper) layer and a reticular (lower) layer. Most of the dermis 18 comprises collagen fibers. However, the dermis also includes various hair bulbs, sweat ducts, sebaceous glands and other glands. The subcutaneous tissue 12 region below the dermis 18 contains fat deposits as well as vessels and other tissue.

In most cases, when applying cosmetic treatment to the skin for tightening or removal of wrinkles, it is desirable to deliver energy to the dermis layer rather than the epidermis, the subcutaneous tissue region 12 or the muscle 14 tissue. In fact, delivery of energy to the subcutaneous tissue region 12 or muscle 14 may produce pockets or other voids leading to further visible imperfections in the skin of a patient. Also, delivery of excessive energy to the epidermis can cause burns and/or scars leading to further visible imperfections.

The application of heat to the fibrous collagen structure in the dermis 18 causes the collagen to dissociate and contract along its length. It is believed that such disassociation and contraction occur when the collagen is heated to about 65 degree C. The contraction of collagen tissue causes the dermis 18 to reduce in size, which has an observable tightening effect. As the collagen contacts, wrinkles, lines, and other distortions become less visible. As a result, the outward cosmetic appearance of the skin 10 improves. Furthermore, the eventual wound healing response may further cause additional collagen production. This latter effect may further serve to tighten and bulk up the skin 10.

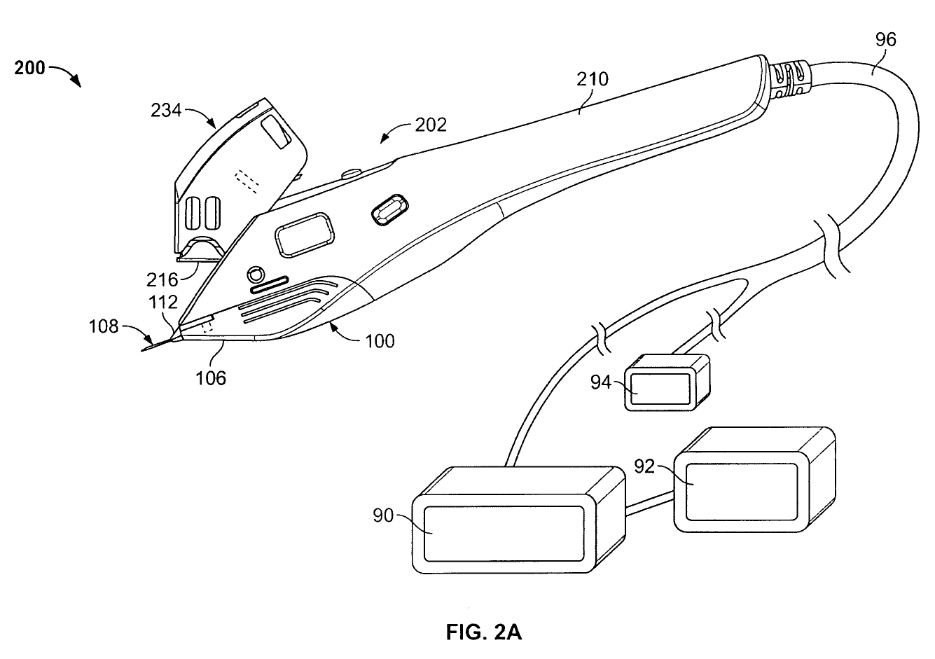

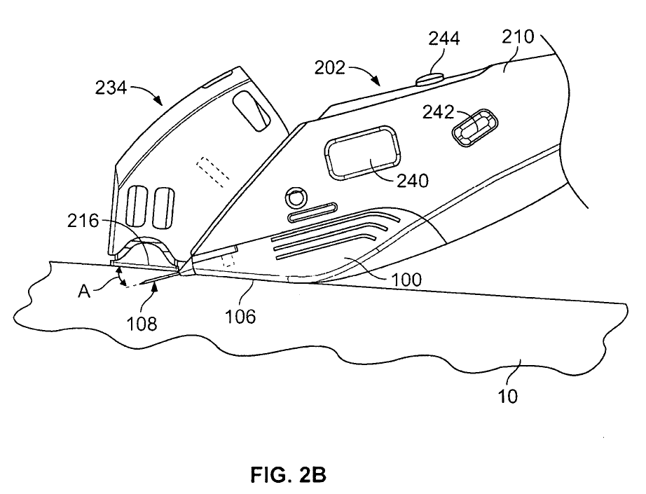

FIG. 2A illustrates a variation of a treatment system according the principles described herein. The treatment system 200 generally includes a treatment unit 202 having a hand-piece or device body 210 (or other member/feature that allows for manipulation of the system to treat tissue 10) that is coupled to a removable cartridge 100. The system 200 shown includes a removable cartridge 100 containing a plurality of retractable energy transfer elements arranged in an array 108. Hereafter, the energy transfer elements are referred to as electrodes 104 and for sake of convenience can include any energy transfer element unless specifically noted otherwise. As shown, the electrodes 104 can optionally extend from a front portion 112 of the cartridge 100. Alternatively, the electrodes 104 can extend from a front face of the device body or from any surface of the device body/cartridge.

The device body 210 is not limited to that shown. Instead, variations include device body shapes that are thinner in profile and can be held at a more vertical angle to the target tissue like a pencil or pointer. Variations also include a device body that has a loop or curved grip that facilitates one specific manner in which it can be grasped by the hand. Any number of variations are possible especially those that ensure the physician's hand does not contact of the distal end of the cartridge or the target tissue.

The devices according to the principles described herein can include any number of arrays depending upon the intended treatment site. Currently, the size of the array, as well as the number of arrays, can change depending on the variation of the invention needed. In most cases, the target region of tissue drives the array configuration. The present invention allows a physician to selectively change array configuration by attaching different cartridges 100. Alternatively, variations of the invention contemplate an electrode assembly that is non-removable from the device body 200.

For example, a treatment unit 202 designed for relatively small treatment areas may only have a single pair of electrodes. On the other hand, a treatment unit 202 designed for use on the cheek or neck may have up to 10 electrode pairs. However, estimates on the size of the electrode array are for illustrative purposes only. In addition, the electrodes on any given array may be the same shape and profile. Alternatively, a single array may have electrodes of varying shapes, profiles, and/or sizes depending upon the intended application.

Furthermore, the array 108 defined by the individual electrodes 104 can have any number of shapes or profiles depending on the particular application. As described in additional detail herein, in those variations of the system 200 intended for skin resurfacing, the length of the electrodes 104 is generally selected so that the energy delivery occurs in the dermis layer of the skin 10 while the spacing of electrodes 104 may be selected to minimize delivery of energy between adjacent pairs of electrodes or to minimize energy to certain areas of tissue.

In those variations where the electrodes 212 are resistive, radiofrequency, microwave, inductive, acoustic, or similar type of energy transfer elements, the electrodes can be fabricated from any number of materials, e.g., from stainless steel, platinum, and other noble metals, or combinations thereof. Additionally, such electrode may be placed on a non-conductive member (such as a polymeric member).

Additionally, the treatment unit 202 may or may not include an actuator as described below for driving the electrode array 108 from the cartridge 100 into the target region. Examples of such actuators include, but are not limited to, gas powered cylinders, springs, linear actuators, or other such motors. Alternative variations of the system 200 include actuators driven by the control system/energy supply unit 90.

FIG. 2A also shows an optional cooling device 234 coupled to the device body 210. The cooling device 234 can be adjustable along the device body 210. In the illustrated variation, the cooling device 234 is in a retracted position where it is spaced away from electrodes 108 (and thus spaced from the surface of the target tissue). This retracted position can aid in the user by allowing for visualization of proper placement of the electrode array 212 into the target tissue. After the user places the device 202 on tissue, the user can advance the cooling device 234 (manually or automatically upon activation of the system) so that a cooling surface 216 of the cooling device 234 makes contact with the target tissue.

The cooling device can be an air or liquid type cooling device. Alternatively, the cooling device can include a Peltier cooling device. A Peltier cooling device can eliminate the need for a fluid source. In some cases, the cooling device can be powered using the same power supply that energizes the electrodes. Such a configuration provides a more compact design that is easier for a medical practitioner to manipulate.

The system 200 also includes an energy supply unit 90 coupled to the treatment unit 202 via a cable 96 or other means. The energy supply unit 90 may contain the software and hardware required to control energy delivery. Alternatively, the CPU, software and other hardware control systems may reside in the hand piece 210 and/or cable 96. It is also noted that the cable 96 may be permanently affixed to the supply unit 90 and/or the treatment unit 202. In additional variations, the hand piece 210 can contain the controls alone or the controls and the power supply necessary to delivery treatment.

In one variation, the energy supply unit 90 may be a RF energy unit. Additional variations of energy supply units may include power supplies to provide thermal energy, ultrasound energy, laser energy, pulsed light energy, and infrared energy. Furthermore, the systems may include combinations of such energy modalities.

For example, in addition to the use of RF energy, other therapeutic methods and devices can be used in combination with RF energy to provide additional or more efficacious treatments. For example, as shown in FIG. 2A, additional energy sources 96 can be delivered via the same or additional energy transfer elements located at the working end of a treatment unit 202. Alternatively, the radiant energy may be supplied by the energy source/supply 90 that is coupled to a diode, fiber, or other emitter at the distal end of the treatment unit 202. In one variation, the energy source/supply 94 and associated energy transfer element may comprise laser, light or other similar types of radiant energy (e.g., visible, ultraviolet, or infrared light). For example, intense pulsed light having a wavelength between 300 and 12000 nm can also be used in conjunction with RF current to heat a targeted tissue. Such associated transfer elements may comprise sources of light at the distal end of the treatment unit 202. These transfer elements may be present on the cartridge 100, on the device, body 210 or even on the cooling unity 234. More specifically a coherent light source or laser energy can be used in conjunction with RF to heat a targeted tissue. Examples of lasers that can be used include erbium fiber, CO₂, diode, flashlamp pumped, Nd:YAG, dye, argon, ytterbium, and Er:YAG among others. More than one laser or light source can be used in combination with RF to further enhance the effect. For example, a pulsed infra-red light source can be used to heat the skin surface, an Nd:YAG laser can be used to heat specific chromophores or dark matter below the surface of the skin, and RF current can be applied to a specific layer within or below the skin; the combination of which provides the optimal results for skin tightening, acne treatment, lipolysis, wart removal or any combination of these treatments.

Other energy modes besides or in addition to the optical energy described above can also be used in conjunction with RF current for these treatments. Ultrasound energy can be delivered either through the RF electrodes, through a face plate on the surface of the skin, or through a separate device. The ultrasound energy can be used to thermally treat the targeted tissue and/or it can be used to sense the temperature of the tissue being heated. A larger pulse of pressure can also be applied to the surface of the skin in addition to RF current to disrupt adipose tissue. Fat cells are larger and their membranes, are not as strong as those of other tissue types so such a pulse can be generated to selectively destroy fat cells. In some cases, the multiple focused pressure pulses or shock waves can be directed at the target tissue to disrupt the cell membranes. Each individual pulse can have from 0.1 to 2.5 Joules of energy.

The energy supply unit 90 may also include an input/output (I/O) device that allows the physician to input control and processing variables, to enable the controller to generate appropriate command signals. The I/O device can also receive real time processing feedback information from one or more sensors associated with the device, for processing by the controller, e.g., to govern the application of energy and the delivery of processing fluid. The I/O device may also include a display, to graphically present processing information to the physician for viewing or analysis.

In some variations, the system 200 may also include an auxiliary unit 92 (where the auxiliary unit may be a vacuum source, fluid source, ultrasound generator, medication source, etc.) Although the auxiliary unit is shown to be connected to the energy supply, variations of the system 200 may include one or more auxiliary units 92 where each unit may be coupled to the power supply 90 and/or the treatment unit 202.

FIG. 2B illustrates a partial view of a working end of a treatment unit 202 where the treatment unit 202 engages against tissue 10 and the array 1(8 extends from a cartridge 100 into the tissue 10. The cooling device 234 also engages tissue 10 so that a cooling surface 216 cools tissue directly above the area of treatment. The illustrated figure also demonstrates another feature of the system where the cartridge 100 includes a tissue engaging surface 106 having a plane that form-s an angle A with a plane of the array of electrodes 108. As described below, this configuration permits a larger treatment area as well as direct cooling of the tissue surface. The devices of the present invention may have an angle A of 15 degrees. However, the angle can range from anywhere between perpendicular to parallel with respect to the tissue surface.

The tissue engaging surface 106 can also include any number of features to ensure adequate contact with tissue.

Although not shown, the tissue engagement surface may contain apertures or other features to allow improved engagement against tissue given the application of a vacuum. By drawing tissue against the tissue engaging surface the medical practitioner may better gauge the depth of the treatment. For example, given the relatively small sectional regions of the epidermis, dermis, and subcutaneous tissue, if a device is placed over an uneven contour of tissue, one electrode pair may be not be placed at the sufficient depth. Accordingly, application of energy in such a case may cause a burn on the epidermis. Therefore, drawing tissue to the tissue engaging surface of the device increases the likelihood of driving the electrodes to a uniform depth in the tissue.

In such an example, the tissue engagement surface 106 can include small projections, barbs, or even an elastic resin to increase friction against the surface of tissue. These projections or features can grip or provide friction relative to the tissue in proximity of the target tissue. This grip or friction holds the tissue in place while the electrodes are inserted at an angle relative to the grip of the projections. In another variation, the tissue engaging surface can include contact or proximity sensors to ensure that any numbers of points along the tissue engaging surface are touching the surface of the target site prior to electrode deployment and/or energy delivery.

FIG. 2B also shows the treatment unit 202 having an extension actuator 240 and a retraction actuator 242 which extend and retract the array 108 in the cartridge. The handle also contains a power control switch 244 that can start and stop delivery of energy. Clearly, the location, size, and construction of such actuators can vary. In addition, all actuators can be replaced by a single actuator. In yet another variation, actuation of the device can occur using a footswitch that is coupled to the control system.

As discussed below, the cooling device 234 includes a cooling plate or cooling surface 216. Optionally, the cooling surface can have a disposable cover that prevents direct tissue contact between the actual cooling surface and the target tissue. The cover can be a disposable, sterilized component that is discarded after each treatment or after each patient.

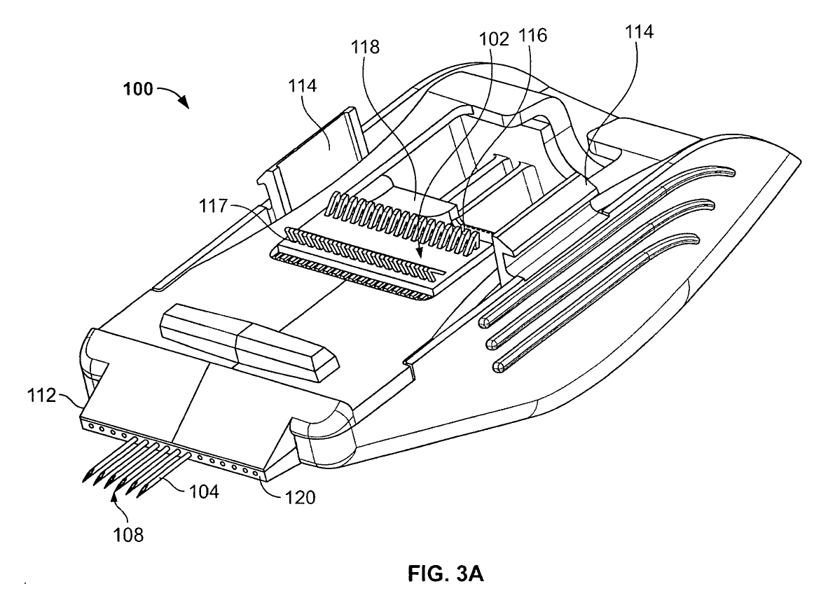

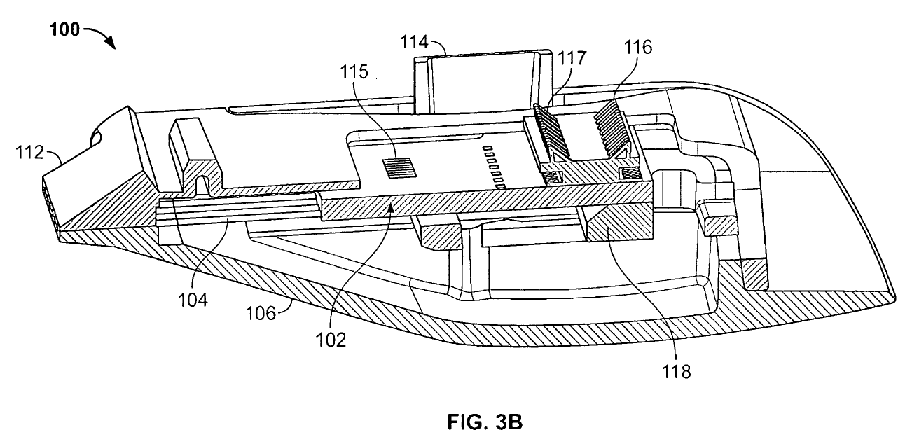

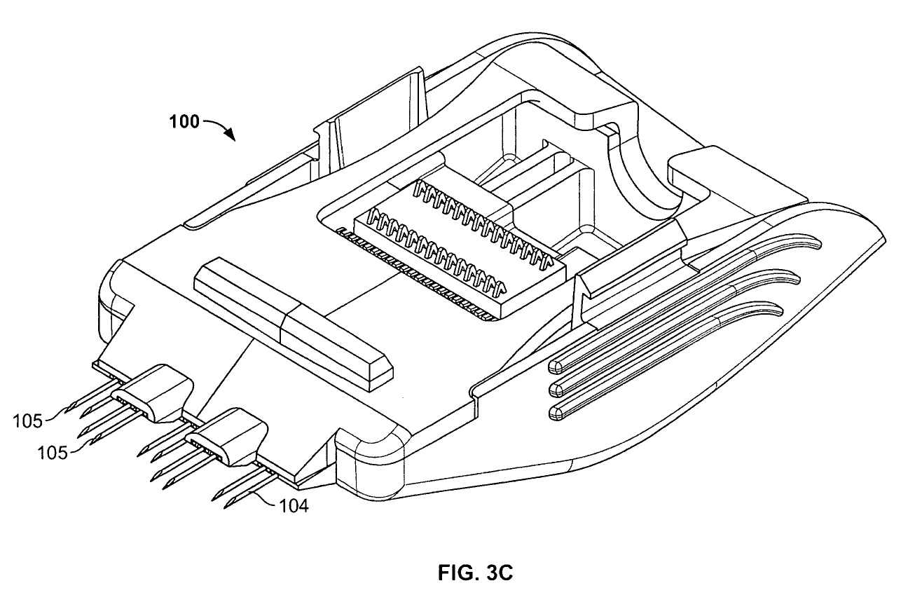

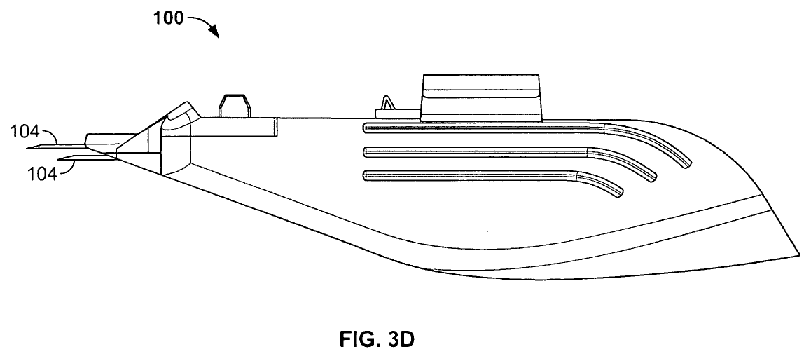

FIG. 3A illustrates one variation of a cartridge body 100 for use with the present system. As shown, the cartridge body 100 includes retention fasteners 114 allowing for coupling with the device body as well as removal from the device body. Again, any number of structures can be incorporated into the device to permit removable coupling of the cartridge body 100 to a treatment unit.

The cartridge body 100 further includes an electrode assembly 102 that is moveable or slidable within the cartridge body 100. The mode of movement of the actuator can include those modes that are used in such similar applications. Examples of these modes include, sliding, rotation, incremental indexing, (via a ratchet-type system), stepping (via an step-motor) Accordingly, the electrode assembly 102 can include a coupling portion or structure 118 that mates with an actuating member in the device body. In the illustrated example, the electrode assembly 102 is in a treatment position (e.g., the array 108 extends from the cartridge 100 allowing for treatment). The electrode assembly 102 includes any number of electrodes 104 that form an array 108 and are extendable and retractable from a portion 104 of the cartridge 100 (as noted above, the electrodes can alternatively extend from the device body, or other parts of the system). As noted above, although the illustrated example shows an array 108 of 1×6 electrodes 104, the array can comprise any dimension of M×N electrodes where the limits are driven by the nature of the treatment site as well as the type of energy delivery required.

FIG. 3A also shows the electrodes 104 in the electrode assembly 102 as having connection or contact portions 116 that couple to a connection board on a treatment unit to provide an electrical pathway from the power supply to the electrodes 104. In the illustrated variation, the electrode assembly 102 as well as the connection portions 116 moves. Such a feature allows for selective connection of the electrodes with the power supply. For example, in certain variations of the system, the electrodes are only coupled to the power supply when in a treatment position and are incapable of delivering energy when in a retracted position. In another variation, the electrode assembly and connection board are configured to permit temperature detection at all times but only energy delivery in the treatment position. Such customization can prevent energy delivery in an unintended location, for example, when the electrodes have an insulation that only allows energy delivery at the distal tip and the intended location of energy delivery is at specific depth in the target tissue that corresponds to the length of the extended electrode the electrode cannot delivery energy to an unintended shallower location when it is not fully extended. However, any number of variations is possible. For example, the system can be configured so that the electrodes can be energized whether in the treatment or retracted positions.

The connection portions 116 can be fabricated in any number of configurations as well. For example, as shown, the connection portions 116 comprise sprint, contacts or spring pins of the type shown. Accordingly, the connection portions 116 can maintain contact with a corresponding contact point trace on a connection board during movement of the electrode assembly 102

FIG. 3A also shows a front portion 112 of the cartridge 100 as having multiple guiding channels 120. These channels 120 can support and guide the electrode 104 as they advance and retract relative to the cartridge 100. The channels 120 can also be configured to provide alternate energy treatments to the surface of the tissue as well as suction or other fluids as may be required by a procedure. One benefit is that a single cartridge design can be configured to support a variety of electrode array configurations. For example rather than the array of six (6) electrodes as shown, the channels 120, can support any number of electrodes (the illustrated example shows a maximum of sixteen (16) but such a number is for exemplary purposes only). Furthermore, the channels 120 need not be only in a linear arrangement as shown, but could be in 1, 2, 3 or more rows or in a random configuration.

FIG. 3B illustrates a cross sectional view of a cartridge 100 when the electrode assembly 102 is in a retracted position. As shown, the connection portions 116, 117 of the electrodes 104 can extend from a top of the electrode assembly 102. The electrode assembly 102 can also optionally include a coupling body 118 to engage an actuator on the treatment device. In this variation, the electrode assembly 102 can have multiple connection portions 116, 117 per individual electrode. In such a case, the multiple connection portions 116 and 117 can be electrically insulated from one another to increase the number of configurations possible with the electrode assembly. For example, and as illustrated below, in one possible variation, the proximal connection portion 116 can electrically couple to a temperature detecting circuit on the hand unit. The distal connection portion 117 can connect to a power delivery circuit only upon distal advancement of the needle assembly 102. In such an example, the temperature of the electrodes can be continuously monitored while the power delivery to the electrodes can be limited to distal advancement of the assembly.

In another aspect of the device, FIG. 3B also shows an example of an electronic memory unit 115, as noted above. The memory unit can provide the system with memory capabilities for containing instructions or record communication between the cartridge and hand unit and/or controller to adjust treatment parameters, monitor usage, monitor sterility, or to record and convey other system or patient characteristics. As also noted above, the unit 115 can also be an RFID antenna or receiver.

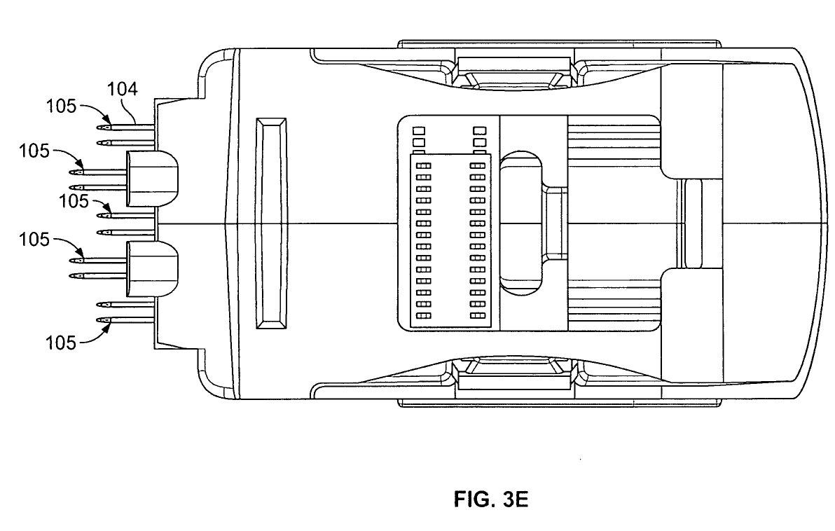

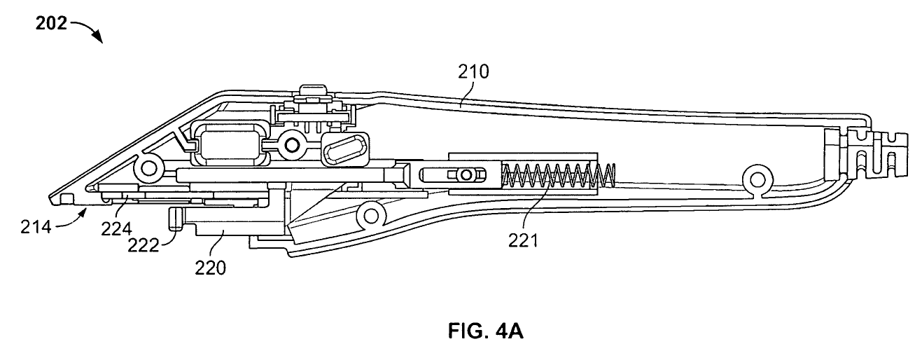

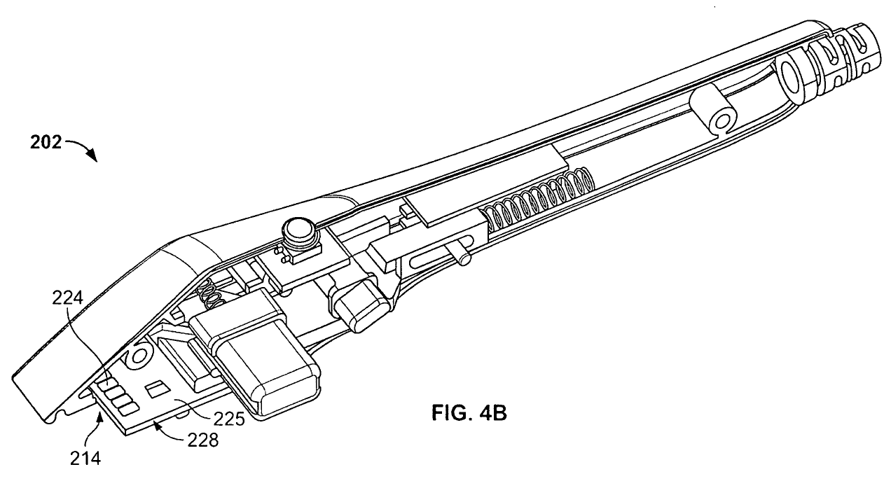

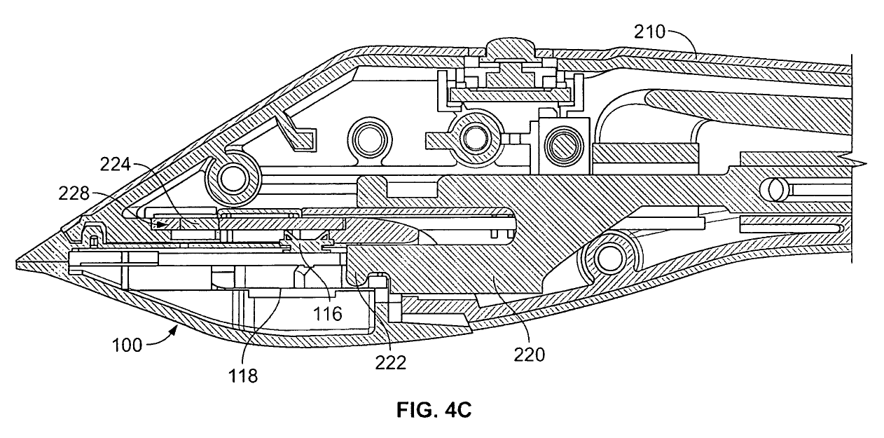

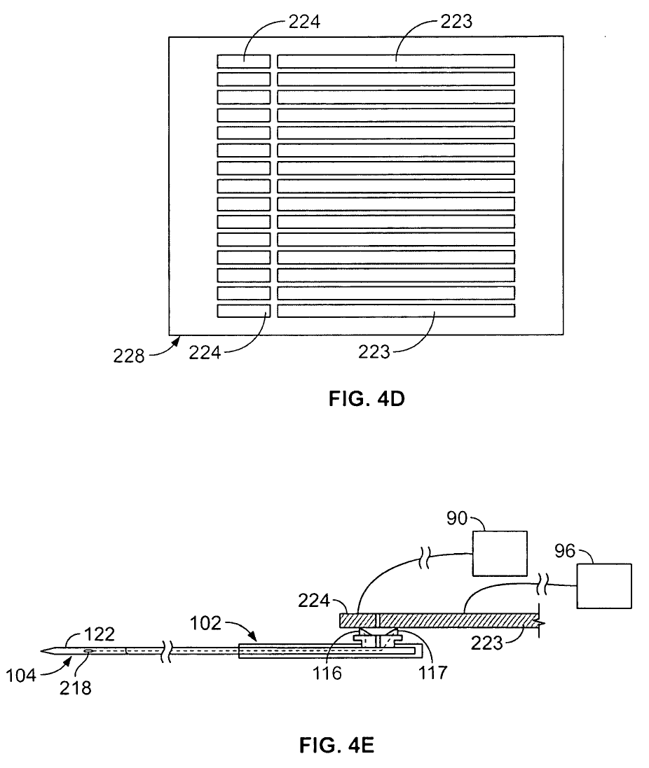

FIG. 4A illustrates a cross sectional view of a variation of a treatment unit 202 without a cartridge attached to a cartridge receiving surface 214 of the device boded 210. As shown, the device body 210 includes a moveable actuator 220 adjacent to the cartridge receiving surface 214. In this variation, the actuator 220 is coupled to a spring 221 such that it may be spring loaded to deliver sufficient force to allow penetration of the array into tissue. However, as noted above, the actuator may comprise any number of actuation means, including, but not limited to, gas powered cylinders, springs, linear actuators, or other such motors. The actuator 220 further includes an engaging surface 222 that can engage a coupler 118 (not shown) on a cartridge assembly.

The cartridge receiving surface 214 further includes a plurality of electrically conductive leads 224 that, when placed in contact with the electrode connection portions discussed above, permit energy delivery via the electrodes in the cartridge.

FIG. 3C shows a perspective view of another variation of an electrode assembly. In this variation, the electrodes 104 are staggered or offset such that adjacent electrode pairs 105 do not form a linear pattern. One such benefit of this configuration is to overcome the creation of a “line effect” in tissue. For example, an array of electrodes arranged in a single line can possibly result in a visible line in tissue defined by the entry points of adjacent and parallel electrodes. In the variation of FIG. 3C, staggering or offsetting the electrodes prevents the “line effect” from occurring.

FIG. 3D shows a side view of the variation of FIG. 3C. As shown, the electrodes 104 are offset to minimize the chance of forming a single continuous line in tissue by penetration of a set of linearly arranged electrodes. Clearly, other configuration can also address the “line effect”. For example, the spacing between adjacent electrodes can be increased to minimize a “line effect” but to still permit efficacy of treatment. In addition, although the illustrated example shows two lines of electrodes, variations of the device include electrodes 104 that form more than two rows of electrodes.

FIG. 3E shows a top view, of the cartridge variation of FIG. 3C. The variation illustrated shows that the plurality of electrodes comprises a plurality of electrode pairs 105. As noted above, the electrode pairs 105 can be vertically offset from an adjacent electrode pair (as shown in FIG. 3D) so that insertion of electrode pairs into the tissue does not create a continuous line of insertion points. Moreover, and as shown in FIG. 3E the electrodes 104 can be axially offset (such that an end of the electrode) extends a greater distance than an end of an adjacent electrode or electrode pair. As noted herein axially offsetting the electrodes allows for a uniform insertion depth when measured relative to a tissue engaging surface of the cartridge.

In one variation, each electrode pair 105 can include an active and return electrode 104 to contain current flow between electrodes in an electrode pair 105. Alternatively, additional configurations are within the scope of the device. For instance, adjacent electrode pairs can serve as opposite poles of a circuit or the electrodes call be monopolar where the therapeutic effect is controlled by selective firing of electrodes. In additional variations, the system can be provided with a number of electrode cartridges where the spacing or offset of the electrodes varies and allows for the physician to control treatment or placement of the electrodes by exchanging cartridges.

FIG. 4B shows a top perspective view of the treatment unit 202 of FIG. 4A. In this view, the back surface of the electrically conductive leads 224 are shown on a circuit or other similar type of connection board 228. It is noted that the wires or other connection means that couple the conductive leads 224 to the power supply are omitted for purposes of illustration. The connection board can also include an electronic memory unit 225, as noted above. Alternatively, the electronic memory unit can be included anywhere in or on the body of the hand unit. The memory unit can provide the system with memory capabilities for containing instructions or record communication between the cartridge and hand unit and/or controller to adjust treatment parameters, monitor usage, monitor sterility, or to record and convey other system or patient characteristics. As also noted above, the unit 225 can also be an RFID antenna or receiver.

FIG. 4C shows a cross sectional view of a device body 210 coupled to a cartridge 100. In the illustration, the electrode assembly is shown in a retracted position such that the electrode connection portions 116 do not contact the electrically conductive leads 224 on the connection board 228. Since the engaging surface 222 and coupling portion 118 move together, distal movement of the actuator 220 causes the electrode assembly and connection portions 116 to move distally towards the treatment position. This movement causes the electrode connection portions 116 to contact the electrically conductive leads 224 allowing for energizing of the electrodes.

As noted above, when provided using a RF energy modality, the ability to control each electrode pair on a separate channel from the power supply provides additional benefits based on the impedance or other characteristic of the tissue being treated. For example, each electrode pair may include a thermocouple to separately monitor each treatment site; the duration of the energy treatment may be controlled depending on the characteristics of the surrounding tissue; selective electrode pairs may be fired rather than all of the electrode pairs firing at once (e.g., by firing electrode pairs that are located on opposite ends of the electrode plate one can further minimize the chance that a significant amount of current flows between the separate electrode pairs.) Naturally, a number of additional configurations are also available depending on the application. Additional variations of the device may include electrode pairs that are coupled to a single channel of a power supply as well.

FIGS. 4D and 4E illustrates a variation of such a configuration. FIG. 4D shows a variation of the underside of a connection board 228 for use in a treatment unit 202. In this variation, the connection board 228 includes a first set of conductive leads 224 and a second set of conductive leads 223 where the first set 224 are electrically isolated from the second set. FIG. 4E illustrates the connection board of FIG. 4D when coupled to an electrode assembly 102. As shown, the electrode 104 in the assembly 102 includes a first and second contact portions 116, 117. When advanced into the treatment position, the first contact portion 116 engages the first conductive lead 224 while the second contact portion 117 engages the second conductive lead 223. In one variation, the first contact portion 224 can provide an electrical pathway to a power supply 90 and the second contact portion 223 can provide an electrical pathway to a temperature measuring unit whether a stand alone unit or a temperature measuring unit coupled to the controller 90. Accordingly, such a configuration permits monitoring of temperature (e.g., via a temperature detecting element 218) continuously but limiting powering of the electrode 104 only when the electrode assembly is in a treatment position (as described herein). Clearly, any number of permutations are within the scope of this disclosure.

The present systems may deliver energy based upon sensing tissue temperature conditions as a form of active process feedback control. Alternatively, the systems may monitor changes in impedance of the tissue being treated and ultimately stop the treatment when a desired value is obtained. In another variation, the delivery of energy can depend on whether impedance is within a certain range. Such impedance monitoring can occur during energy delivery and attenuate power if the dynamically measured impedance starts to exceed a given value or if the rate of increase is undesirably high. Yet another mode of energy delivery is to provide a total maximum energy over a duration of time.

As noted herein, temperature or other sensing may be measured beneath the epidermis in the dermis region. Each probe or electrode may include a sensor or the sensor may be placed on a structure that penetrates the tissue but does not function as an energy delivery electrode. In yet another variation, the sensors may be a vertically stacked array (i.e. along the length of the electrode) of sensors to provide data along a depth or length of tissue.

Energizing the RF electrodes in the dermal layer produces a healing response caused by thermally denaturing the collagen in the dermal layer of a target area. As noted herein, systems according to the present invention are able to provide a desirable effect in the target area though they use a relatively low amount of energy when compared to systems that treat through the epidermis. Accordingly, systems of the present invention can apply energy in various modes to improve the desired effect at the target area.

In one mode, the system can simply monitor the amount of energy being applied to the target site. This process involves applying energy and maintaining that energy at a certain pre-determined level. This treatment can be based on a total amount of energy applied and/or application of a specific amount of energy over a set period of time. In addition, the system can measure a temperature of the target site during the treatment cycle and hold that temperature for a pre-determined amount of time. However, in each of these situations, the system does not separate the time or amount of energy required to place the target site in the desired state from the time or amount of energy required to hold the target site in the desired state. As a result, the time or amount of energy used to place the target in a desired state (e.g., at a pre-determined temperature) is included in the total treatment cycle. In some applications it may be desirable to separate the portion of the treatment cycle required to elevate the target to a pre-determined condition from the portion of the treatment cycle that maintains the target site at the pre-determined conditions.

For example, in one variation, the system can maintain a temperature of the target site at a pre-determined treatment temperature during a pre-determined cycle or dwell time. The system then delivers energy to maintain the target site at the treatment temperature. Once the target site reaches the treatment temperature, the system then maintains this condition for the cycle or dwell time. This variation allows for precise control in maintaining the target site at the pre-determined temperature. In another variation, the system can monitor the amount of power applied to the target site for a specific dwell time. By continuously measuring current and output voltage, the system can calculate both the impedance changes and the delivered power levels. With this method a specific amount of power can be delivered to the target tissue for a specified amount of time. In addition, the above variations can be combined with various methods to control time, temperature or energy parameters to place the tissue in the desired state. For example, the system can employ a specified ramp time or maximum energy to achieve the pre-determined treatment temperature. Such a variation can create a faster or slower ramp to the treatment temperature.

Although the treatment of tissue generally relies on energy to affect the tissue, the mere act of inserting the electrode array into tissue can also yield therapeutic benefits. For instance, the mechanical damage caused by placement of the electrodes also produces an adjunct healing response. The healing response to injury in the skin tissue can contribute to the production of new collagen (collagenesis) that can further improve the tone or appearance of the skin. Accordingly, in one variation a medical practitioner may opt to use the methods and systems to create mechanical injury to tissue by placing electrodes into target areas without thermal treatment to induce, a healing response in the targeted area. Accordingly, the invention is not limited to application of energy via the electrodes.

The low energy requirements of the system present an additional advantage since the components on the system undergo less stress than those systems needing higher amounts of energy. In those systems requiring higher energy, RF energy is often delivered in a pulsed fashion or for a specific duty cycle to prevent stressing the components of that system. In contrast, the reduced energy requirements of the present system allow for continual delivery of RF energy during a treatment cycle. In another variation, the duty cycle of variations of the present system can be pulsed so that temperature measurements can be taken between the pulsed deliveries of energy. Pulsing the energy delivery allows for an improved temperature measurement in the period between energy deliveries and provides precise control of energy delivery when the goal of the energy delivery is to reach a pre-determined temperature for a pre-determined time.

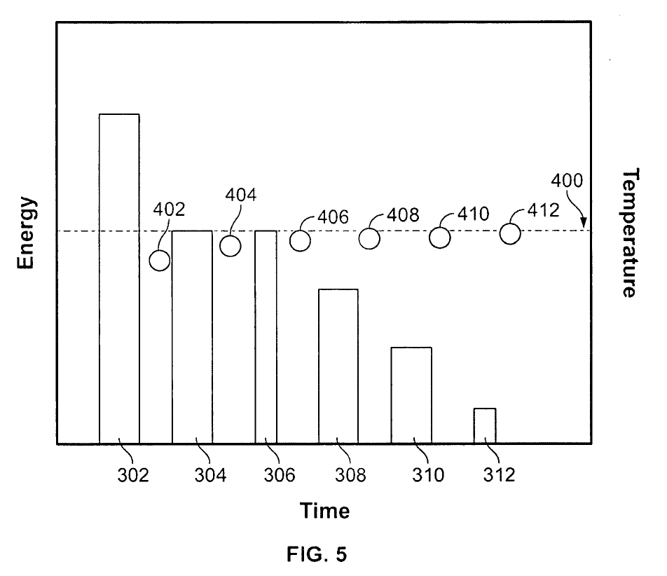

FIG. 5 illustrates a graph of energy delivery and temperature versus time. As shown, the pulses or cycles of energy are represented by the bars 302, 304, 306, 308, 310, 312. Each pulse has a parameter, including amount of energy, duration, maximum energy delivered, energy wave form or profile (square wave, sinusoidal, triangular, etc), current, voltage, amplitude, frequency, etc. As shown in the graph, measurements are taken between pulses of energy. Accordingly, between each pulse of energy delivery one or more temperature sensor(s) near the electrode obtains a temperature measurement 402, 404, 406, 408, 410, 412. The controller compares the measured temperature to a desired temperature (illustrated by 400). Based on the difference, the energy parameters are adjusted for the subsequent energy pulse. Measuring temperature between pulses of energy allows for a temperature measurement that is generally more accurate than measuring during the energy delivery pulse. Moreover, measuring between pulses allows for minimizing the amount of energy applied to obtain the desired temperature at the target region.

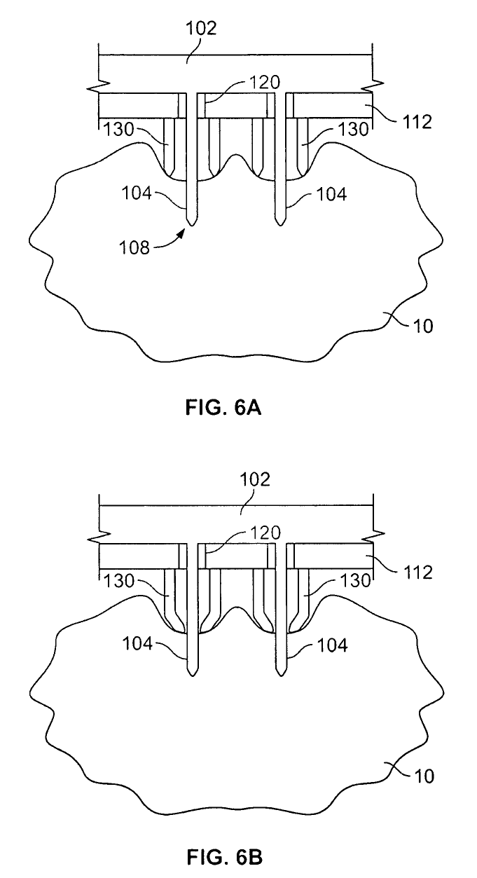

FIG. 6A illustrates an aspect for use with the variations of the devices described herein. In this example, the electrodes 104 advance through an introducer member or cannula 130 located on the front face 112 of a cartridge. The cannula 130 places tissue 10 in a state of tension (also called “traction”). In this variation the introducer/cannula 130 is located about each channel 120 in the cartridge.

As shown, once the introducer member 130 engages tissue 10, the tissue first elastically deforms as shown. Eventually, the tissue can no longer deflect and is placed in traction by the introducer members 130. As a result, the electrodes 104 more readily penetrate the tissue.

FIG. 6B illustrates another variation of the introducer member 130 that is tapered inwards toward the electrodes so that the opening at the distal end closely fits around the electrode.

In another variation, insertion of the array 108 can consist of 2 or more steps. In the first step the actuation of the extension presses the channels 120 against, the target tissue to create a state of traction. Further actuation advances the array 108 through the channels 120 and into the target tissue. Since the target tissue is under traction, the array requires less force to penetrate the tissue. In another variations, the channels 120 can be individual cannula that extend from the distal face of the cartridge. Such a configuration produces traction on a smaller portion of target tissue. Alternatively, the two step extension process can be composed of a first step which extends small projections out of the tissue engaging surface of the cartridge in a direction that is substantially opposite of the direction of electrode extension which occurs in the second step. This alternative creates more traction which further eases insertion of the electrodes as the target tissue is stretched in opposite directions.

In those variations of the device using an RF energy modality, the electrodes 104 can be arranged in a pair configuration. In a bi-polar configuration one electrode serves a first pole, while the second electrode serves as the second pole (it is also common to refer to such electrodes as the active and return electrodes). The spacing of electrode pairs is sufficient so that the pair of electrodes is able to establish a treatment current path therebetween for the treatment of tissue. However, adjacent electrode pairs can be spaced sufficiently to minimize the tendency of current flowing between the adjacent pairs. Typically, each electrode pair is coupled to a separate power supply or to a single power supply having multiple channels for each electrode pair.

The benefit of such a configuration is that, when compared to conventional treatments, the amount of power required to induce heating in the target tissue is much reduced. For example, because the electrodes are spaced to provide heating across the electrode pairs at the target tissue, each channel of the system may provide as little as 1 watt of energy to produce the desired temperature increase at the site. In additional variations, the amount of energy may be no more than 3 or 5 watts. However, any amount of energy necessary to accomplish the desired effect is within the scope of this invention. In contrast, if a treatment system delivered energy over the entire electrode array a much greater amount of energy is required to generate the desired temperature over the larger surface area of tissue. Moreover, the energy demand is less because the treatment applies energy directly to the target tissue rather than though additional layers of tissue.

In one variation of the device, it is believed that a desirable spacing of the first and second electrode poles is between 1 and 3 mm, while a desirable spacing of electrode pairs is between 5 and 6 mm. In one example, the described configuration allowed for each independent channel to deliver no more than 1 watt, 3 watts, 5, watts or any other amount of energy to deliver acceptable tissue treatment results. Obviously, the power supply may be configured to deliver greater amounts of energy as needed, depending on the application.

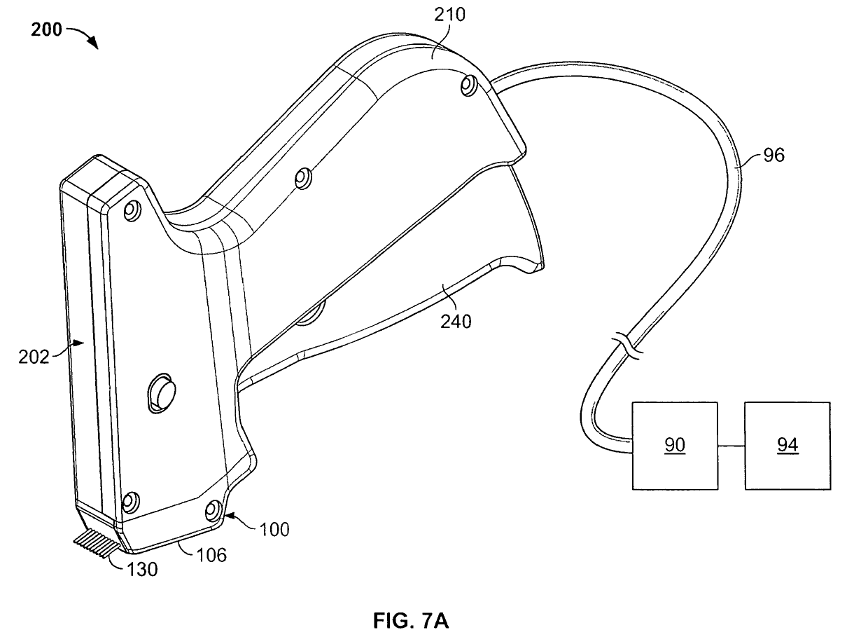

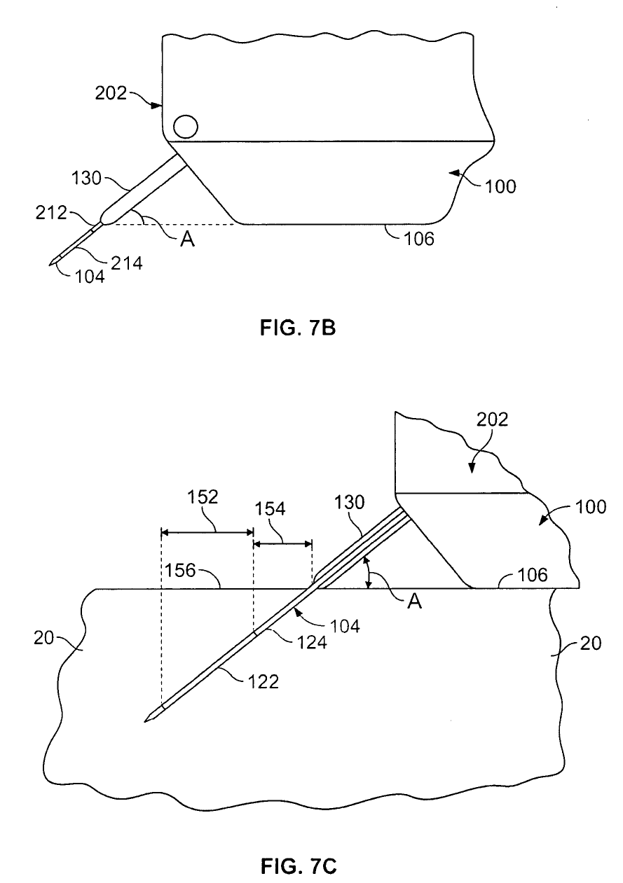

FIG. 7A illustrates another variation of a system 200 for use in accordance with the principles discussed herein. In this variation, the system 200 includes a treatment unit 202 having a cartridge 100 from which a cannula or introducer member 130 extend at an oblique angle relative to a tissue engagement surface 106. As described below, the ability to insert the electrodes (not shown) into the tissue at an oblique angle increases the treatment area and allows for improved cooling at the tissue surface. Although the variation only shows a single array of introducers for electrodes, variations of the invention may include multiple arrays of electrodes. In addition, the devices and systems described below may be combined with the features described herein to allow for improved penetration of tissue. The devices of the present invention may have an angle. A of 15 degrees. However, the angle may be anywhere from ranging between 5 and 85 degrees.

Although the introducer member 130 is shown as being stationary, variations of the device include introducer members that are slidable on the electrodes. For example, to ease insertion of the electrode, the electrode may be advanced into the tissue. After the electrode is in the tissue, the introducer member slides over the electrode to a desired location. Typically, the introducer member is insulated and effectively determines the active region of the electrode. In another variation using RF energy, the introducer member may have a return electrode on its tip. Accordingly, after it advances into the tissue, application of energy creates current path between the electrode and the return electrode on the introducer.

The treatment unit 202 of the device 200 may also include a handle portion 210 that allows the user to manipulate the device 200. In this variation, the handle portion 210 includes a lever or lever means 240 that actuates the electrodes into the tissue (as discussed in further detail below).

As discussed above, the device 200 can be coupled to a power supply 90 with or without an auxiliary unit 94 via a connector or coupling member 96. In some variations of the device, a display or user interface can be located on the body of the device 200 as discussed below.

FIG. 7B illustrates a partial side view of the electrodes 104 and tissue engaging surface 106 of the electrode device of FIG. 7A. As shown, the electrodes 104 extend from the cartridge 100 through the introducer 130. In alternate variations, the electrodes can extend directly from the body of the device or through extensions on the device.

As shown, the electrodes 104 are advanceable from the cartridge (in this case through the introducers 130) at an oblique angle A as measured relative to the tissue engagement surface 106. The tissue engagement surface 106 allows a user to place the device on the surface of tissue and advance the electrodes 104 to the desired depth of tissue. Because the tissue engagement surface 106 provides a consistent starting point for the electrodes, as the electrodes 104 advance from the device 202 they are driven to a uniform depth in the tissue.

For instance, without a tissue engagement surface, the electrode 104 may be advanced too far or may not be advanced far enough such that they would partially extend out of the skin. As discussed above, either case presents undesirable outcomes when attempting to treat the dermis layer for cosmetic affects. In cases where the device is used for tumor ablation, inaccurate placement may result in insufficient treatment of the target area.

FIG. 7C illustrates a magnified view of the electrode entering tissue 20 at an oblique angle A with the tissue engaging surface 106 resting on the surface of the tissue 20. As is shown, the electrode 104 can include an active area 122. Generally, the term “active area” refers to the part of the electrode through which energy is transferred to, or, from the tissue. For example, the active area could be a conductive portion of an electrode, it can be a resistively heated portion of the electrode, or even comprise a window through which energy transmits to the tissue. Although this variation shows the active area 122 as extending over a portion of the electrode, variations of the device include electrodes 104 having larger or smaller active areas 122.

In any case, because the electrodes 104 enter the tissue at an angle A, the resulting region of treatment 152, corresponding to the active area 122 of the electrode is larger than if the needle were driven perpendicular to the tissue surface. This configuration permits a larger treatment area with fewer electrodes 104. In addition, the margin for error of locating, the active region 122 in the desired tissue region is greater since the length of the desired tissue region is greater at angle A than if the electrode were deployed perpendicularly to the tissue.

As noted herein, the electrodes 104 may be inserted into the tissue in either a single motion where penetration of the tissue and advancement into the tissue are part of the same movement or act. However, variations include the use of a spring mechanism or impact mechanism to drive the electrodes 104 into the tissue. Driving the electrodes 104 with such a spring-force increases the momentum of the electrodes as they approach tissue and facilitates improved penetration into the tissue. As shown below, variations of the devices discussed herein may be fabricated to provide for a dual action to insert the electrodes. For example, the first action may comprise use of a spring or impact mechanism to initially drive the electrodes to simply penetrate the tissue. Use of the spring force or impact mechanism to drive the electrodes may overcome the initial resistance in puncturing the tissue. The next action would then be an advancement of the electrodes so that they reach their intended target site. The impact mechanism may be spring driven, fluid driven or via other means known by those skilled in the art. One possible configuration is to use an impact or spring mechanism to fully drive the electrodes to their intended depth.

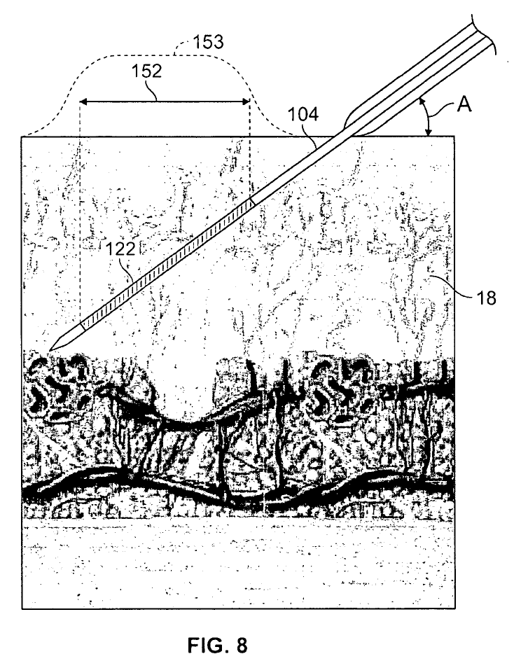

FIG. 8 illustrates an example of the benefit of oblique entry when the device is used to treat the dermis 18. As shown, the length of the dermis 18 along the active region 122 is greater than a depth of the dermis 18. Accordingly when trying to insert the electrode in a perpendicular manner, the shorter depth provides less of a margin for error When trying to selectively treat the dermis region 18. As discussed herein, although the figure illustrates treatment of the dermis to tighten skin or reduce wrinkles, the device and methods may be used to affect skin anomalies 153 such as acne, warts sebaceous glands, tattoos, or other structures or blemishes. In addition the electrode may be inserted to apply energy to a tumor, a hair follicle, a fat layer, adipose tissue, SMAS, a nerve or a pain fiber or a blood vessel.

Inserting the electrode at angle A also allows for direct cooling of the surface tissue. As shown in FIG. 7C, the area of tissue on the surface 156 that is directly adjacent or above the treated region 152 (i.e., the region treated by the active area 122 of the electrode 104) is spaced from the entry point by a distance or gap 154. This gap 154 allows for direct cooling of the entire surface 156 adjacent to the treated region 152 without interference by the electrode or the electrode mounting structure. In contrast, if the electrode were driven perpendicularly to the tissue surface, then cooling must occur at or around the perpendicular entry point.

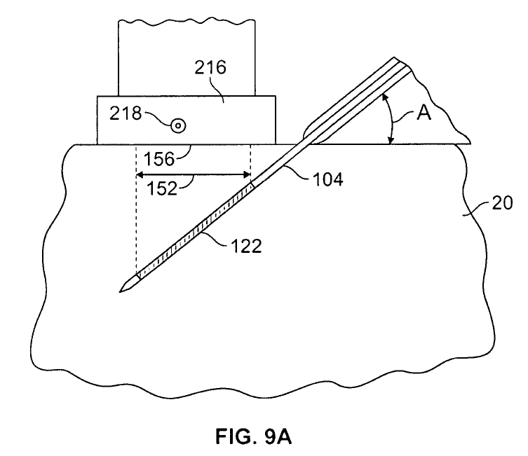

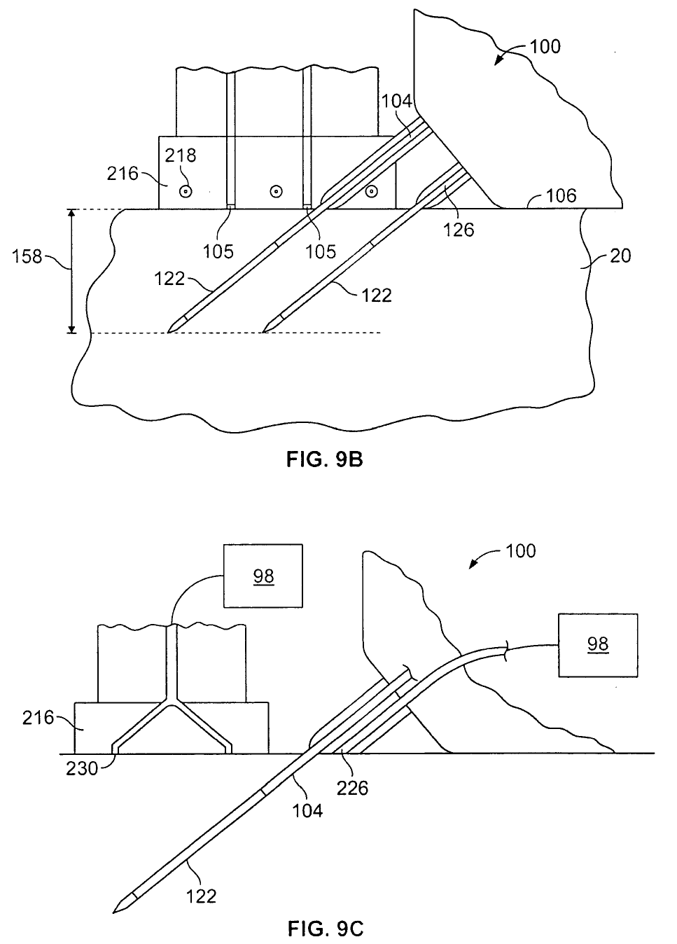

FIG. 9A illustrates one example of a cooling surface 216 placed on body structure or tissue 20. As shown, the electrode 104 enters at an oblique angle A such that the active region 122 of the electrode 104 is directly adjacent or below the cooling surface 216. In certain variations, the cooling surface 216 may extend to the entry point (or beyond) of the electrode 104. However, it is desirable to have the cooling surface 216 over the electrode's active region 122 because the heat generated by the active region 122 will have its greatest effect on the surface at the surface location 156. In some variations, devices and methods described herein may also incorporate a cooling source in the tissue engagement surface.

The cooling surface 216 and cooling device may be any cooling mechanism known by those skilled in the art. For example, it may be a manifold type block having liquid or gas flowing through for convective cooling. Alternatively, the cooling surface 216 may be cooled by a thermoelectric cooling device (such as a fan or a Peltier-type cooling device). In such a case, the cooling may be driven by energy from the electrode device thus eliminating the need for additional fluid supplies. One variation of a device includes a cooling surface 216 having a temperature detector 218 (thermocouple, RTD, optical measurement, or other such temperature measurement device) placed within the cooling surface. The device may have one or more temperature detectors 218 placed anywhere throughout the cooling surface 216 or even at the surface that contacts the tissue.

In one application, the cooling surface 216 is maintained at or near body temperature. Accordingly, as the energy transfer occurs causing the temperature of the surface 156 to increase, contact between the cooling surface 216 and the tissue 20 shall cause the cooling surface to increase in temperature as the interface reaches a temperature equilibrium. Accordingly, as the device's control system senses an increase in temperature of the cooling surface 216 additional cooling can be applied thereto via increased fluid flow or increased energy supplied to a Peltier-type device. The cooling surface can also pre-cool the skin and underlying epidermis prior to delivering the therapeutic treatment. Alternatively, or in combination, the cooling surface can cool the surface and underlying epidermis during and/or subsequent to the energy delivery where such cooling is intended to maintain the epidermis at a specific temperature below that of the treatment temperature. For example the epidermis can be kept at 30 degrees C. when the target tissue is raised to 65 degrees C.

When treating the skin, it is believed that the dermis should be heated to a predetermined temperature condition, at or about 65 degree C., without increasing the temperature of the epidermis beyond 42 degree C. Since the active area of the electrode designed to remain beneath the epidermis, the present system applies energy to the dermis in a targeted, selective fashion, to dissociate and contract collagen tissue. By attempting to limit energy delivery to the dermis, the configuration of the present system also minimizes damage to the epidermis.

While the cooling surface may comprise any commonly known thermally conductive material, metal, or compound (e.g., copper, steel, aluminum, etc.). Variations of the devices described herein may incorporate a translucent or even transparent cooling surface. In such cases, the cooling device will be situated so that it does not obscure a view of the surface tissue above the region of treatment.

In one variation, the cooling surface can include a single crystal aluminum oxide (Al₂O₃). The benefit of the single crystal aluminum oxide is a high thermal conductivity optical clarity, ability to withstand a large temperature range, and the ability to fabricate the single crystal aluminum oxide into various shapes. A number of other optically transparent or translucent substances could be used as well (e.g., diamond, other crystals or glass).

FIG. 9B illustrates another aspect for use with variations of the devices and methods described herein. In this variation, the cartridge 100 includes two arrays of electrodes 104, 126. As shown, the first plurality 104 is spaced evenly apart from and parallel to the second plurality 126 of electrodes. In addition, as shown, the first set of electrodes 104 has a first length while the second set of electrodes 126 has a second length, where the length of each electrode is chosen such that the sets of electrodes 104, 126 extend into the tissue 20 by the same vertical distance or length 158. Although only two arrays of electrodes are shown, variations of the invention include any number of arrays as required by the particular application. In some variations, the lengths of the electrodes 104, 126 are the same. However, the electrodes will be inserted or advanced by different amounts so that their active regions penetrate a uniform amount into the tissue. As shown, the cooling surface may include more than one temperature detecting element 218.

FIG. 9BB also illustrates a cooling surface 216 located above the active regions 122 of the electrodes. In such a variation, it may be necessary for one or more of the electrode arrays to pass through a portion of the cooling surface 216. Alternative variations of the device include electrodes that pass through a portion of the cooling device (such as the Peltier device described below).

FIG. 9BB also show a variation of the device having additional energy transfer elements 105 located in the cooling surface 216. As noted above, these energy transfer elements can include sources of radiant energy that can be applied either prior to the cooling surface contacting the skin, during energy treatment or cooling, or after energy treatment

FIG. 9C shows an aspect for use with methods and devices of the invention that allows marking of the treatment site. As shown, the cartridge 100 may include one or more marking lumens 226, 230 that are coupled to a marking ink 98. During use, a medical practitioner may be unable to see areas once treated. The use of marking allows the practitioner to place a mark at the treatment location to avoid excessive treatments. As shown, a marking lumen 226 may be placed proximate to the electrode 104. Alternatively, or in combination, marking may occur at or near the cooling surface 216 since the cooling surface is directly above the treated region of tissue. The marking lumens may be combined with or replaced by marking pads. Furthermore, any type of medically approved dye may be used to mark. Alternatively, the dye may comprise a substance that is visible under certain wavelengths of light. Naturally, such a feature permits marking and visualization by the practitioner given illumination by the proper light source but prevents the patient from seeing the dye subsequent to the treatment.

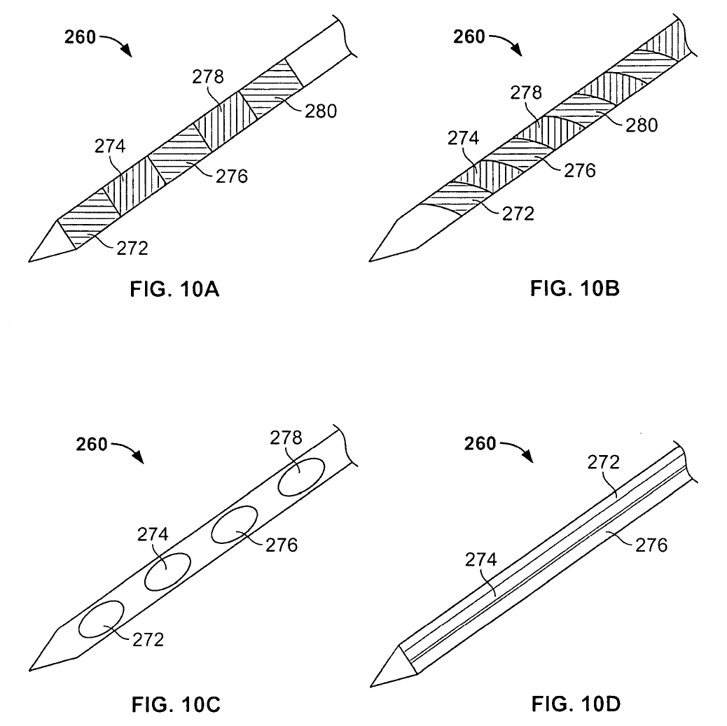

FIGS. 10A-10D illustrate variations of electrodes for use with the systems and methods described herein. Depending upon the application, it may be desirable to provide an electrode 260 that has a variable resistance along the active region of the electrode 260. FIGS. 10A-10D illustrate a partial example of such electrodes. As shown in FIGS. 10A and 10B, an electrode may have concentric or spiral bands that create varying ranges of impedance 272, 274, 276, 278, and 280 along the electrode 260. In addition, as shown in FIG. 10C, the electrode 260 may have regions 272, 274, and 276 and 278 along the electrode of varying resistance. FIG. 10D illustrates a similar concept where the regions of resistance 272, 274, and 276, run in longitudinal stripes along the electrode 260. These configurations may be fabricated through spraying, dipping, plating, anodizing, plasma treating, electro-discharge, chemical applications, etching, etc.

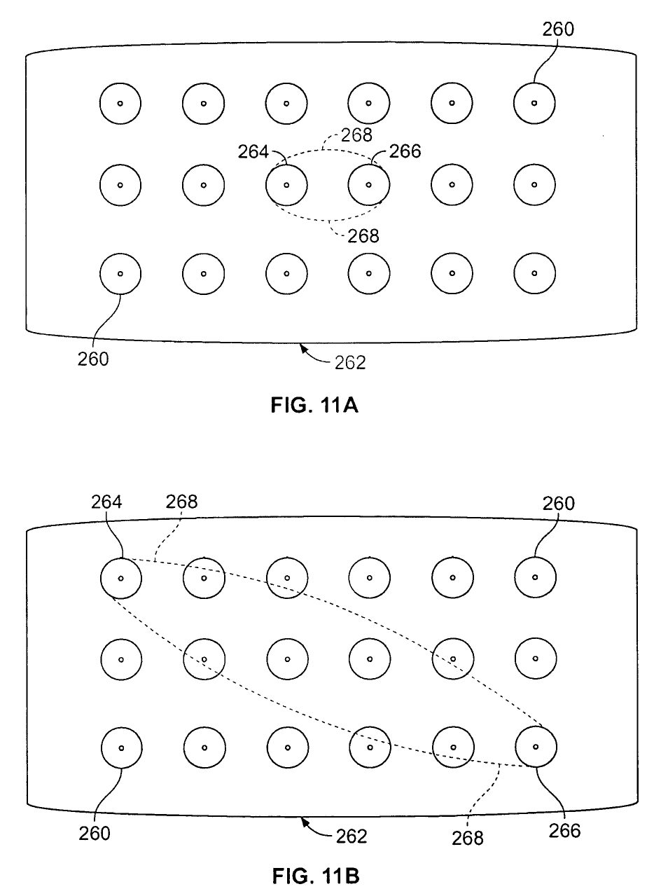

FIGS. 11A-11B illustrate examples of system configurations that can be incorporated into any conventional electrode array or into the devices described above using RF energy. As shown, in this example the electrode array 262 comprises 3×6 array of electrode. However, the selective pairing of electrodes can occur with a 1×N array as well as an M×N array. Each electrode in the array 262 is configured to energize separately. This configuration provides the ability of any given pair of electrodes to form a circuit for treating tissue. In one example, in the variation of FIG. 11A, the power supply energizes adjacent electrode pairs 264, 266. This configuration generates the smallest treatment area in the electrode array 262. FIG. 11B illustrates a situation where the farthest electrode pairs 264, 266 within the array 262 are triggered to form a current path 268. One benefit of this configuration is that a single electrode array may form a number of patterns based on various combinations of pairs that may be formed in the array. The array may be able to provide a denser treatment or more uniform tissue heating. The treatment can deliver targeted therapy to key areas, of tissue. In one variation, various pairs of the electrode array may be triggered sequentially during a single insertion.

Although the systems described herein may be used by themselves the invention includes the methods and devices described above in combination with substances such as moisturizers, ointments, etc. that increase the resistivity of the epidermis. Accordingly, prior to the treatment, the medical practitioner can prepare the patient by increasing the resistivity of the epidermis. During the treatment, because of the increased resistivity of the epidermis, energy would tend to flow in the dermis.

In addition, such substances can be combined with various other energy delivery modalities to provide enhanced collagen production in the targeted tissue or other affects as described herein.

In one example, 5-aminolevulinic acid (ALA) or other photolabile compounds that generate a biologically active agent when present in the skin upon exposure to sunlight or other applied spectrums of activating light. Coatings or ointments can also be applied to the skin surface in order to stabilize the soft tissue. Temporarily firming or stabilizing the skin surface will reduce skin compliance and facilitate the insertions of the electrodes of the current device. An agent such as cyanoacrylate spirit gum, latex, a facial mask or other substance that cures into a rigid or semi-rigid layer can be used to temporarily stabilize the skin. The topical ointments or coatings can be applied to enhance collagen production or to stabilize the skin for ease of electrode insertion or both. Furthermore, topical agents can be applied to alter the electrical properties of the skin. Applying an agent which increases the impedance of the epidermal layer will reduce the conductance of RF current through that layer and enhance the conductance in the preferred dermal layer. A topical agent that penetrates the epidermal layer and is absorbed by the dermal layer can be applied that lowers the impedance of the dermal layer, again to enhance the conduction of RF current in the dermal, layer. A topical agent that combines both of these properties to affect both the dermal and epidermal layers conductance can also be used in combination with RF energy delivery.

In addition to topical agents, the invention with its use of penetrating electrodes lends itself to the delivery of agents and materials directly to a specific region of tissue. For example, anesthetic agents such as lidocaine can be delivered through the electrode cannula to the dermis and epidermis to deaden nerve endings prior to the delivery of therapeutic energy. Collagen or other filler material can be delivered prior to, during or after energy delivery. Botulinum Toxin Type A, Botox, or a similar neurotoxic can be delivered below the skin layer to create temporary paralysis of the facial muscles after energy delivery. This maybe provide a significant improvement in the treatment results as the muscles would not create creases or wrinkles in the skin while the thermally treated collagen structure remodeled and collagenesis occurs.

Another means to enhance the tissue's therapeutic response is the use of mechanical energy through massage. Such an application of mechanical energy can be combined, with the methods and systems described herein. Previously, devices have used massaging techniques to treat adipose tissue. For example, U.S. Pat. No. 5,961,475 discloses a massaging device that applies negative pressure as well as massage to the skin. Massage both increases blood circulation to the tissue and breaks done connections between the adipose and surrounding tissue. For example, these effects combined with energy treatment of the tissue to enhance the removal of fat cells.

The above variations are intended to demonstrate the various examples of embodiments of the methods and devices of the invention. It is understood that the embodiments described above may be combined or the aspects of the embodiments may be combined in the claims.

What is claimed is:

1. An energy delivery device for applying energy from an energy source to treat a target region beneath a surface of tissue, the device comprising:
a device body having a handle portion, a cartridge receiving surface, an actuator adjacent thereto and a plurality of electrically conductive leads on at least a portion of the cartridge receiving surface and being electrically coupleable to the energy source, where the actuator is moveable relative to the device body;
a cartridge body removably coupled to the device body on the cartridge receiving surface, the cartridge body comprising an electrode assembly in engagement with the actuator, the electrode assembly having a plurality of electrodes arranged in an array and where at least one electrode has a connection portion, the electrode assembly being moveable between a treatment position and a retracted position upon movement of the actuator, such that in the treatment position the at least one electrode extends from the cartridge body and the respective connection portion engages one electrically conductive lead, and in the retracted position, the at least one electrode retracts into the cartridge and the respective connection portion moves out of engagement with the electrically conductive lead preventing delivery of energy.

2. The energy delivery device of claim 1, the cartridge body further comprising a tissue, engaging surface, where a plane of the tissue engaging surface forms an oblique angle relative to a plane of the array of the electrodes, such that when extended the from the cartridge body the electrodes extend at the oblique angle relative to the tissue engaging surface, where placement of the tissue engaging surface against tissue allows for entry of the electrodes into the tissue at the oblique angle.

3. The energy delivery device of claim 1, the plurality of electrodes comprises a first plurality of electrodes arranged in a first array, and a second plurality of electrodes arranged in a second array.

4. The energy delivery device of claim 3, where the first plurality of electrodes comprise a first length, and the second plurality of electrodes comprise a second length, where the first and second length are not equal such that upon insertion into tissue, each plurality of needles extends a same vertical length into the tissue.

5. The energy delivery device of claim 3, where at least one of the first plurality of electrodes are offset from the second plurality of electrodes such that upon insertion into tissue, the plurality of electrodes does not create a continuous line of insertion points along the plurality of electrodes.

6. The energy delivery device of claim 1, the plurality of electrodes comprises a plurality of electrode pairs, where at least one electrode pair is vertically offset from an adjacent electrode pair so that insertion of electrode pairs into the tissue does not create a continuous line of insertion points.

7. The energy delivery device of claim 6, where at least one of the electrode pairs is axially offset from an adjacent electrode pair.

8. The energy delivery device of claim 1, where the actuator is coupled to a spring means and is spring loaded.

9. The energy delivery device of claim 8, where the spring means comprises a spring force when restrained and when unrestrained applies the spring force to the actuator to drive electrode assembly from the retracted position to the treatment position with sufficient force for the electrodes to penetrate the surface of the tissue.

10. The energy delivery device of claim 1, where the actuator is slidable or rotatable relative to the device body.

11. The energy delivery device of claim 1, where the device body is configured to allow single handed deployment of the actuator.

12. The energy delivery device of claim 1, where the actuator is coupled to a compressed fluid cylinder, having a valve for driving the actuator.

13. The energy delivery device of claim 1, where a number of the conductive leads is greater than a number of the electrodes.

14. The energy delivery device of claim 1, where at least one of the electrodes comprises a temperature detecting element.

15. The energy delivery device of claim 14, where the plurality of electrically conductive leads comprise a first set of energy leads and a second set of temperature detecting leads, and where the energy leads and the temperature detecting leads are electrically isolated from each other.

16. The energy delivery device of claim 14, where each connection portion of the electrode comprises a energy connection portion and a temperature detecting connection portion, and where in the treatment position, the energy connection portion contacts at least one energy lead and the temperature detecting portion contacts one temperature detecting lead.

17. The energy delivery device of claim 16, where the temperature detecting portion also contacts the temperature detecting, lead in the retracted portion.

18. The energy delivery device of claim 14, where the temperature detecting element is configured to detect a temperature of the electrode in both the retracted position and the treated position.

19. The energy delivery device of claim 1, where the device body further comprises a cooling surface removably coupled adjacent to the cartridge receiving surface such that when the electrode array is in the treatment position and the electrodes are inserted into tissue, the cooling surface is positioned to engage an area of the tissue surface directly above the electrodes, and where the cooling surface is adapted to maintain a temperature at, below, or slightly above body temperature.

20. The energy delivery device of claim 19, where the cooling surface is visually transparent.

21. The energy delivery device of claim 19, where the cooling surface is visually translucent.

22. The energy delivery device of claim 19, where the cooling surface comprises a material selected from the group consisting of steel, aluminum, and copper.

23. The energy delivery device of claim 19, where the plurality of electrodes pass through a portion of the cooling surface when advanced from the device body.

24. The energy delivery device of claim 19, further comprising a thermoelectric cooling device coupled to the power supply and in contact with the cooling surface, where the thermoelectric cooling device maintains the temperature.

25. The energy delivery device of claim 24, where the plurality of electrodes pass through a portion of the thermoelectric cooling device when advanced from the device body.

26. The energy delivery device of claim 24, where the thermoelectric cooling device comprises a Peltier cooling device.

27. The energy delivery device of claim 19 further comprising a fluid source coupled to the cooling surface, where the fluid source is adapted to maintain the temperature.

28. The energy deliver), device of claim 1, where each of the electrodes has a respective impedance along a respective active region, and wherein the impedance of at least one of the electrodes varies along its respective active region.

29. The energy delivery device of claim 28, where the impedance of each of the plurality of electrodes varies along the respective active region.

30. The energy delivery device of claim 1, where at least one electrode in the plurality of electrodes comprises a section of insulation over a portion of an active region.

31. The energy delivery device of claim 1, where the device body further comprises an ink-pad having ink and located on an exterior surface, where the ink pad marks tissue upon contact therewith.

32. The energy delivery device of claim 1, where the device body further comprises a marking lumen for spraying an ink on an exterior surface of the tissue.

33. The energy delivery device of claim 1, where the cartridge body further comprises a marking lumen for spraying an ink on an exterior surface of the tissue.

34. The energy delivery device of claim 1, where the tissue engaging surface further comprises at least one sensor for detecting contact with the surface of tissue.

35. A kit for applying therapeutic energy to tissue, the kit comprising:
a device body having a handle portion, a cartridge receiving surface, an actuator adjacent thereto and a plurality of electrically conductive leads on at, least a portion of the cartridge receiving surface and being electrically coupleable to the energy source;
a plurality of cartridge bodies, each cartridge body capable of being removably coupled to the device body on the cartridge receiving surface, each cartridge body comprising an electrode assembly in engagement with the actuator, the electrode assembly having a plurality of electrodes having an active region and arranged in an array and at least one electrode having a connection portion, the electrode assembly being moveable between a treatment position and a retracted position upon movement of the actuator, such that in the treatment position the at least one electrode extends from the cartridge body and the respective connection portion contacts one electrically conductive lead, and in the retracted position the at least one electrode retracts into the cartridge and the respective connection portion moves out of engagement with the electrically conductive lead preventing delivery of energy, and
where the electrode assembly on each respective cartridge body has a different configuration.

36. The kit of claim 35, where a number of electrodes in a first cartridge body is different from a length of the electrodes in a second cartridge body.

37. The kit of claim 35, where a length of the electrodes in a first cartridge body is different from a length of the electrodes in a second cartridge body.

38. The kit of claim 35, where a gauge of the electrodes in a first cartridge body is different from a gauge of the electrodes in a second cartridge body.

39. The kit of claim 35, where the active region of the electrodes in a first cartridge body is different from the active region of the electrodes in a second cartridge body.

40. The kit of claim 35, where the plurality of electrodes in at least a first cartridge body comprises a first plurality of electrodes arranged in a first array, and a second plurality of electrodes arranged in a second array.

41. The kit of claim 40, where at least one of the first plurality of electrodes are offset from the second plurality of electrodes such that upon insertion into tissue, the plurality of electrodes does not create a continuous line of insertion points along the plurality of electrodes.

42. The kit of claim 41, where a second cartridge body comprises a first plurality and a second plurality of electrodes where the first and second plurality of electrodes are offset, and where the offset of the first cartridge body is different from the offset of the second cartridge body.

43. The kit of claim 35, where the plurality of electrodes on a first cartridge body comprises a plurality of electrode pairs, where at least one electrode pair is vertically offset from an adjacent electrode pair so that insertion of electrode pairs into the tissue does not create a continuous line of insertion points.

44. The kit of claim 43, where at least one of the electrode pairs is axially offset from an adjacent electrode pair.

45. The kit of claim 35, where a spacing between the electrodes in a first cartridge body is different from the spacing between the electrodes in a second cartridge body.

46. A method for applying energy to a region of tissue from a energy source, comprising:
placing a tissue engaging surface of an electrode device against a surface of the tissue, where the electrode device comprises a plurality of electrodes having a retracted position within the electrode device and a treatment position in which the electrodes extend from the treatment, device at an oblique angle relative to the tissue engaging surface, at least one electrode including an active region at a distal portion thereof, wherein the electrodes are decoupled from the energy source when in the retracted position;
deploying the electrodes to the treatment positions in which the electrodes are coupled to the energy source;
applying energy to tissue at the active region of the electrode.

47. The method of claim 46, where at least a portion of the electrodes are offset from an adjacent electrode such that insertion of the electrodes into tissue does not create a continuous line of insertion points.

48. The method of claim 46, where the plurality of electrodes comprise a set of electrodes and where the set of electrodes are detachable from the electrode device.

49. The method of claim 48, further comprising replacing the plurality of electrodes with a second set of electrodes, where at least one feature of the second set of electrodes is chosen based on the region of tissue

50. The method of claim 46, further comprising placing a cooling surface adjacent to an entry point of the electrodes, where the cooling surface directly cools the exterior surface of the tissue directly above the active region of the electrode.

51. The method of claim 46, where the cooling surface is visually transparent.

52. The method of claim 46, where the cooling surface is visually translucent.

53. The method of claim 46, where the cooling surface comprises a material selected from the group consisting of steel, aluminum, and copper.

54. The method of claim 46, where the plurality of electrodes pass through a portion of the cooling surface when inserting the electrodes into the tissue.

55. The method of claim 46, where inserting the plurality of electrodes comprises first applying an impact force to the electrodes such that the electrodes penetrate the tissue and subsequently, advancing the electrodes farther into the tissue.

56. The method of claim 46, further comprising:
placing a portion of the surface, of the tissue that is adjacent to each electrode in a state of traction as the electrodes are inserted into the tissue;
advancing at least one of the electrodes through the surface layer;
applying energy to at least one of the electrodes to create a thermal injury to tissue beneath the surface layer.

57. The method of claim 56, where each electrode extends through an opening in an introducer member, where pressing the introducer member against the surface layer places the surface layer in traction.

58. The method of claim 56, further comprising a power supply configured to energize adjacent electrodes at different times.

59. The method of claim 46, where the region of tissue comprises tissue selected from the dermis, a tumor, a hair follicle, sebaceous gland, a skin anomaly such as acne, wart or other structure or blemish, and a blood vessel.

60. An energy delivery device for applying energy from an energy source to treat a target region beneath a surface of tissue, the device comprising:
a device body having a handle portion, a cartridge receiving surface, a spring-loaded actuator adjacent thereto and a plurality of electrically conductive leads on at least a portion of the cartridge receiving surface and being electrically coupleable to the energy source;
a cartridge body removably coupled to the device body on the cartridge receiving surface, the cartridge body comprising an electrode assembly in engagement with the actuator, the electrode assembly having a plurality of electrodes arranged in an array and each electrode having a connection portion, the electrode assembly being moveable between a treatment position and a retracted position upon movement of the actuator, such that in the treatment position each electrode extends from the cartridge body and the respective connection portion contacts one electrically conductive lead, and in the retracted position, each electrode retracts into the cartridge and the respective connection portion moves out of engagement with the electrically conductive lead preventing delivery of energy.

61. The energy delivery device of claim 60, the cartridge body further comprising a tissue engaging surface, where a plane of the tissue engaging surface forms an oblique angle relative to a plane of the array of the electrodes, such that when extended the form the cartridge body the electrodes extend at the oblique angle relative to the tissue engaging surface, where placement of the tissue engaging surface against tissue allows for entry of the electrodes into the tissue at the oblique angle.
